(19) United States
(12) Patent Application Publication
(10) Pub. No.: US 2010/0292345 A1
(43) Pub. Date: Nov. 18, 2010

(54) THERAPEUTIC USES OF CANNABIGEROL

(75) Inventor: Roger Pertwee, Aberdeen (GB)

Correspondence Address: WOLF GREENFIELD & SACKS, P.C., 600 ATLANTIC AVENUE, BOSTON, MA, 02210-2206, US

(73) Assignee: GW PHARMA LIMITED, SALISBURY WILTSHIRE (GB)

(21) Appl. No.: 12/666,385

(22) PCT Filed: Jun. 25, 2008
(86) PCT No.: PCT/GB08/02179

§ 371 Date: Jun. 7, 2010

(30) Foreign Application Priority Data
Jun. 25, 2007 (GB) 0712303.7

(51) Int. Cl. A61K 31/05; C07C 39/10; A61P 25/00; A61P 25/28; A61P 25/16; A61P 25/08; A61P 9/10; A61P 29/00; A61P 37/00; A61P 3/00; A61P 11/00; A61P 31/00; A61P 31/06; A61P 37/08; A61P 11/06; A61P 17/06; A61P 35/00; A61P 3/10; A61P 19/10; A61P 1/08

(52) U.S. Cl. 514/733; 568/766

(57) ABSTRACT

The present invention relates to the use of the cannabinoid cannabigerol (CBG) in the manufacture of medicaments for use in the treatment of diseases and conditions benefiting from concurrent agonism of the CBi and the CB2 cannabinoid receptors. Such diseases or conditions to be treated are taken from the group: pain, neurodegenerative disease, ischemic disease, brain injury or damage, acquired brain injury, age related inflammatory or autoimmune disease, cachexia, nausea and vomiting, glaucoma, movement disorders, rheumatoid arthritis, asthma, allergy, psoriasis, Crohn's disease, systemic lupus erythematosus, diabetes, cancer, osteoporosis, renal ischemia and nephritis.

The present invention relates to the use of the cannabinoid cannabigerol (CBG) in the manufacture of medicaments for use in the treatment of diseases and conditions benefiting from concurrent agonism of the CB₁ and the CB₂ cannabinoid receptors.

### BACKGROUND TO THE INVENTION

The action of many known cannabinoids can be attributed to their interaction with cannabinoid receptors. Cannabinoid receptors are present in mammalian systems and several classes of G-Protein coupled receptors have been identified. The receptors that are present mainly in the central nervous system are known as CB₁ receptors, whereas a different type of receptor, which are found substantially in the immune system, are known as the CB₂ receptors.

Cannabinoids are generally known to be cannabinoid receptor agonists. When a cannabinoid receptor agonist binds to a cannabinoid receptor a response is triggered. This response is known as a signalling pathway.

Compounds which are known to bind to the CB₁ cannabinoid receptor include delta-9-tetrahydrocannabinol (THC), R-(+)-WIN55212 and anandamide. These compounds are as such described as CB₁ agonists as when they bind to the CB₁ receptor a specific response is produced.

Agonism at a receptor will often lead to an active response by the cell. Many diseases or conditions can be alleviated by the administration of cannabinoid receptor agonists.

Such diseases and conditions include but are not limited to the following: pain (including but not limited to acute pain; chronic pain; neuropathic pain and cancer pain), neurodegenerative disease (including but not limited to Alzheimer's disease; Parkinson's disease; amyotrophic lateral sclerosis; Huntington's disease; multiple sclerosis; frontotemporal dementia; prion disease; Lewy body dementia; progressive supranuclear palsy; vascular dementia; normal pressure hydrocephalus; traumatic spinal cord injury; HIV dementia; alcohol induced neurotoxicity; Down's syndrome; epilepsy or any other related neurological or psychiatric neurodegenerative disease), ischemic disease (including but not limited to stroke; cardiac ischemia; coronary artery disease; thromboembolism; myocardial infarction or any other ischemic related disease), brain injury or damage (including but not limited to traumatic brain injury is taken from the group: diffuse axonal injury; concussion; contusion; whiplash or any other traumatic head or brain injury), acquired brain injury (including but not limited to stroke; anoxic brain injury; hypoxic brain injury or any other acquired brain injury), age related inflammatory or autoimmune disease, cachexia (including related conditions such as AIDS wasting disease, weight loss associated with cancer, chronic obstructive pulmonary disease or infectious diseases such as tuberculosis), nausea and vomiting, glaucoma, movement disorders, rheumatoid arthritis, asthma, allergy, psoriasis, Crohn's disease, systemic lupus erythematosus, diabetes, cancer, osteoporosis, renal ischemia and nephritis.

The diseases and conditions listed above may all benefit from agonism of either the CB₁ and/or the CB₂ cannabinoid receptor. Due to the multifactorial nature of many of these diseases and conditions it is credible to suppose that agonism at one or more of the receptors may be beneficial in their treatment.

The ability of a compound to have agonist properties concurrently at both the CB₁ and CB₂ receptors may be of great use clinically.

It is known that agonism of the CB₁ receptor in man can cause side effects, for example a study on the use of dronabinol in clinical trials for AIDS-related wasting disease reported the following side effects: asthenia, palpitations, tachycardia, vasodilation, facial flush, abdominal pain, nausea, vomiting, amnesia, anxiety, nervousness, confusion, depersonalisation, dizziness, euphoria, hallucination, paranoia, somnolence and abnormal thinking.

The CB₂ receptor is highly localized in the immune cells and as such agonism at these receptors produces a regulation of immune function and inflammatory pain.

It is thought that concurrent agonism at both the CB₁ and CB₂ receptors might attenuate the side effects caused by direct agonism of the CB₁ receptor.

It has previously been shown that CBG, along with many other natural and synthetic cannabinoids is a CB₁ receptor agonist as described by US 2007/0060638. This application describes the use of cannabinoid receptor agonists in combination with a cannabinoid receptor antagonist for use in the treatment of drug or alcohol addictions.

In addition the topical use of cannabinoid extracts as an analgesic has been described in U.S. Pat. No. 6,949,582. The cannabinoid extract of the patent includes all of the naturally occurring cannabinoids, terpenes and flavinoids that are found in cannabis plant extracts. Amongst these is the cannabinoid cannabigerol.

Surprisingly the applicants have shown that the cannabinoid cannabigerol (CBG) is an agonist of both the CB₁ and CB₂ cannabinoid receptors.

The cannabinoid CBG is a non-psychoactive phytocannabinoid and as such has the dual benefits of being both able to concurrently agonise both CB₁ and CB₂ receptors whilst not causing the psychoactive side effects of other commonly used cannabinoids such as THC.

CBG is a naturally occurring cannabinoid and is a precursor to the major cannabinoids CBD, CBC and THC and as such is rarely found in cannabis plants in any significant concentration. As such this cannabinoid was not thought to possess pharmacological properties making this finding even more surprising.

### SUMMARY OF THE INVENTION

According to the first aspect of the present invention there is provided the use of the cannabinoid cannabigerol (CBG) in the manufacture of a medicament for use in the treatment of diseases and conditions benefiting from agonism of the CB₁ and/or the CB₂ cannabinoid receptors.

Preferably the cannabinoid cannabigerol (CBG) is used in the manufacture of a medicament for use in the treatment of diseases and conditions benefiting from agonism of the CB₁ cannabinoid receptor.

Alternatively the cannabinoid cannabigerol (CBG) is used in the manufacture of a medicament for use in the treatment of diseases and conditions benefiting from agonism of the CB₂ cannabinoid receptor.

More preferably the cannabinoid cannabigerol (CBG) is used in the manufacture of a medicament for use in the treatment of diseases and conditions benefiting from concurrent agonism of the CB₁ and the CB₂ cannabinoid receptors.

Preferably the diseases or conditions to be treated are taken from the group: pain (including but not limited to acute pain; chronic pain; neuropathic pain and cancer pain), neurodegenerative disease (including but not limited to Alzheimer's disease; Parkinson's disease; amyotrophic lateral sclerosis; Huntington's disease; multiple sclerosis; frontotemporal dementia; prion disease; Lewy body dementia; progressive supranuclear palsy; vascular dementia; normal pressure hydrocephalus; traumatic spinal cord injury; HIV dementia; alcohol induced neurotoxicity; Down's syndrome; epilepsy or any other related neurological or psychiatric neurodegenerative disease), ischemic disease (including but not limited to stroke; cardiac ischemia; coronary artery disease; thromboembolism; myocardial infarction or any other ischemic related disease), brain injury or damage (including but not limited to traumatic brain injury is taken from the group: diffuse axonal injury; concussion; contusion; whiplash or any other traumatic head or brain injury), acquired brain injury (including but not limited to stroke; anoxic brain injury; hypoxic brain injury or any other acquired brain injury), age related inflammatory or autoimmune disease, cachexia (including related conditions such as AIDS wasting disease, weight loss associated with cancer, chronic obstructive pulmonary disease or infectious diseases such as tuberculosis), nausea and vomiting, glaucoma, movement disorders, rheumatoid arthritis, asthma, allergy, psoriasis, Crohn's disease, systemic lupus erythematosus, diabetes, cancer, osteoporosis, renal ischemia and nephritis.

References to CBG, CBG type compounds or derivatives thereof, particularly with regard to therapeutic use, will be understood to also encompass pharmaceutically acceptable salts of such compounds. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

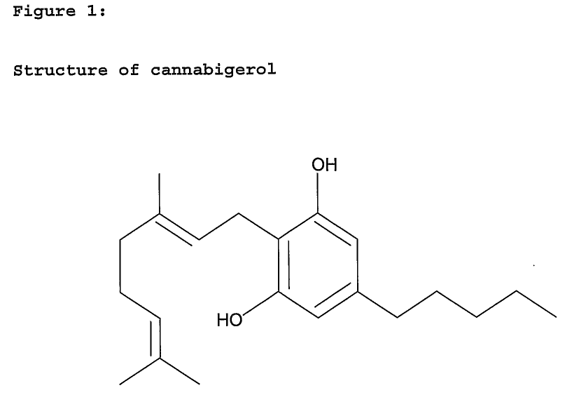

Cannabinoid biosynthesis begins when a precursor molecule reacts with geranylpyrophosphate to form a ringed structure. As shown in FIG. 1, CBG type compounds are mostly 21 carbon compounds.

Variation in the length of the side chain that is attached to the aromatic ring (bottom right hand side of the structure) can produce different types of CBG compounds. For example when the side chain is a pentyl (5 carbon) chain the compound produced will be CBG. If the pentyl chain is replaced with a propyl (3 carbon) chain the CBD type compound formed is CBGV (cannabigeroldivarin). The propyl variant will be formed if a 10 carbon precursor is reacted at the first stage of the biosynthetic pathway rather than a 12 carbon compound.

Synthetic variants of CBG include dimethylheptyl CBG. This variant also has variations in the side chain of the CBG compound.

The scope of the invention also extends to derivatives of CBG that retain the desired activity of concurrent agonism of the CB₁ and CB₂ receptors. Derivatives that retain substantially the same activity as the starting material, or more preferably exhibit improved activity, may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutical active agents such as, for example, improved solubility, reduced toxicity, enhanced uptake, etc.

The term concurrent is understood herein to refer to simultaneous and essentially independent binding of cannabigerol to the CB₁ and CB₂ receptors.

Preferably the cannabigerol is in the form of an extract prepared from at least one cannabis plant.

More preferably the extract from at least one cannabis plant is a botanical drug substance.

Preferably the extract from at least one cannabis plant is produced by extraction with supercritical or subcritical CO₂.

Alternatively the extract from at least one cannabis plant is produced by contacting plant material with a heated gas at a temperature which is greater than 100° C., sufficient to volatilise one or more of the cannabinoids in the plant material to form a vapour, and condensing the vapour to form an extract.

Preferably the extract from at least one cannabis plant comprises all of the naturally occurring cannabinoids in the plant.

Alternatively the cannabigerol is in a substantially pure or isolated form.

A “substantially pure” preparation of cannabinoid is defined as a preparation having a chromatographic purity (of the desired cannabinoid) of greater than 90%, more preferably greater than 95%, more preferably greater than 96%, more preferably greater than 97%, more preferably greater than 98%, more preferably greater than 99% and most preferably greater than 99.5%, as determined by area normalisation of an HPLC profile.

Preferably the substantially pure cannabigerol used in the invention is substantially free of any other naturally occurring or synthetic cannabinoids, including cannabinoids that occur naturally in cannabis plants. In this context “substantially free” can be taken to mean that no cannabinoids other than the active cannabigerol are detectable by HPLC.

In another aspect of the present invention cannabigerol is in a synthetic form.

Preferably the cannabigerol is formulated as a pharmaceutical composition further comprising one or more pharmaceutically acceptable carriers, excipients or diluents.

The invention also encompasses pharmaceutical compositions comprising CBG type compound or derivative thereof, or pharmaceutically acceptable salts or derivatives thereof, formulated into pharmaceutical dosage forms, together with suitable pharmaceutically acceptable carriers, such as diluents, fillers, salts, buffers, stabilizers, solubilisers, etc. The dosage form may contain other pharmaceutically acceptable excipients for modifying conditions such as pH, osmolarity, taste, viscosity, sterility, lipophilicity, solubility etc. The choice of diluents, carriers or excipients will depend on the desired dosage form, which may in turn be dependent on the intended route of administration to a patient.

Suitable dosage forms include, but are not limited to, solid dosage forms, for example tablets, capsules, powders, dispersible granules, cachets and suppositories, including sustained release and delayed release formulations. Powders and tablets will generally comprise from about 5% to about 70% active ingredient. Suitable solid carriers and excipients are generally known in the art and include, e.g. magnesium carbonate, magnesium stearate, talc, sugar, lactose, etc. Tablets, powders, cachets and capsules are all suitable dosage forms for oral administration.

Liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Liquid dosage forms also include solutions or sprays for intranasal, buccal or sublingual administration. Aerosol preparations suitable for inhalation may include solutions and solids in powder form, which may be combined with a pharmaceutically acceptable carrier, such as an inert compressed gas.

Also encompassed are dosage forms for transdermal administration, including creams, lotions, aerosols and/or emulsions. These dosage forms may be included in transdermal patches of the matrix or reservoir type, which are generally known in the art.

Pharmaceutical preparations may be conveniently prepared in unit dosage form, according to standard procedures of pharmaceutical formulation. The quantity of active compound per unit dose may be varied according to the nature of the active compound and the intended dosage regime. Generally this will be within the range of from 0.1 mg to 1000 mg.

According to a second aspect of the present invention there is provided a method for the treatment or prevention of diseases benefiting from concurrent agonism of the CB₁ and the CB₂ cannabinoid receptors, which comprises administering to a subject in need thereof an effective amount of cannabigerol.

### SPECIFIC DESCRIPTION

There are over sixty identified cannabinoids that are known to be produced the by cannabis plant. Of these cannabinoids there are eight different main classes of cannabinoids: cannabigerol-type; cannabichromene-type; cannabidiol-type; tetrahydrocannabinol-type; cannabielsoin-type; iso-tetrahydrocannabinol-type; cannabicyclol-type; and cannabicitran-type.

All of these main classes of cannabinoids are derived from cannabigerol-type compounds and differ mainly in the way the CBG precursor is cyclised.

The structure of cannabigerol is shown in FIG. 1. Cannabinoid production in cannabis plants begins when an enzyme causes geranyl pyrophosphate and olivetolic acid to condense to form cannabigerol. The CBG cannabinoid is then usually converted by cannabinoid synthase enzymes to cannabidiol (CBD), cannabichromene (CBC) or tetrahydrocannabinol (THC).

Due to the nature of the biosynthetic pathway of cannabinoids most cannabis plants do not comprise a large amount of CBG. As such the pharmacology of CBG is largely unknown and it has been postulated that CBG is merely a precursor to other more pharmacologically active cannabinoids.

Due to the biosynthetic pathway of the cannabinoids, it is possible that CBG will share some common properties with its products such as CBD and CBC. Also it is highly conceivable that the combination of CBG with it's products such as CBC, CBD and THC will produce a greater and more beneficial effect than that produced by CBG alone.

It was shown by Elsohly et al. in 1992 that CBG had antimicrobial properties and more recently in 2005 Maor et al. described a synthetic analogue of CBG, CBG-dimethyl heptyl which possessed hypotensive and vasorelaxant properties. Additionally the applicant's co-pending patent application (US 60/813814) describes the antidepressant properties of cannabigerol. Compared with the vast knowledge available on THC or CBD, CBG's properties are relatively unknown.

Some patients have found cannabis to be useful in the treatment of many different diseases or conditions ranging from multiple sclerosis, glaucoma and nausea. However reports on the therapeutic potential of cannabis are often contradictory as they describe the effects of whole, usually smoked cannabis, rather than the actions of the specific cannabinoids themselves.

The example detailed below describes studies undertaken to investigate the properties of CBG at the CB₁ and CB₂ cannabinoid receptors. In particular the ability of CBG to bind to CB₁ and CB₂ receptors was investigated.

Certain aspects of this invention are further described, by way of example only, with reference to the accompanying drawings in which:

FIG. 1 shows the structure of cannabigerol;

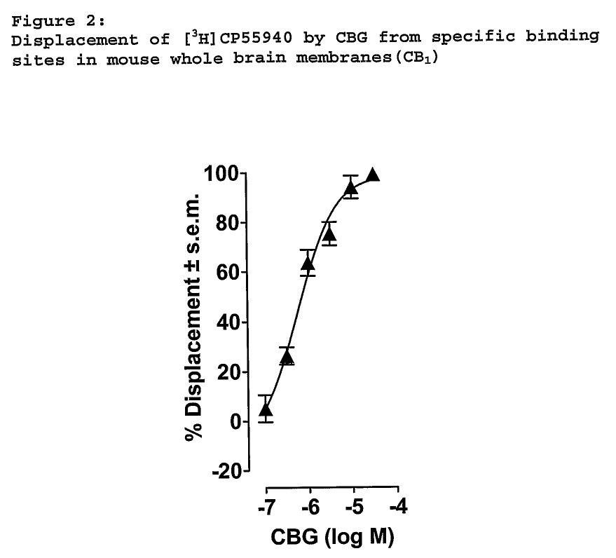

FIG. 2 shows a graph of displacement of [³H]CP55940 by CBG from specific binding sites in mouse whole brain membranes (CB₁); and

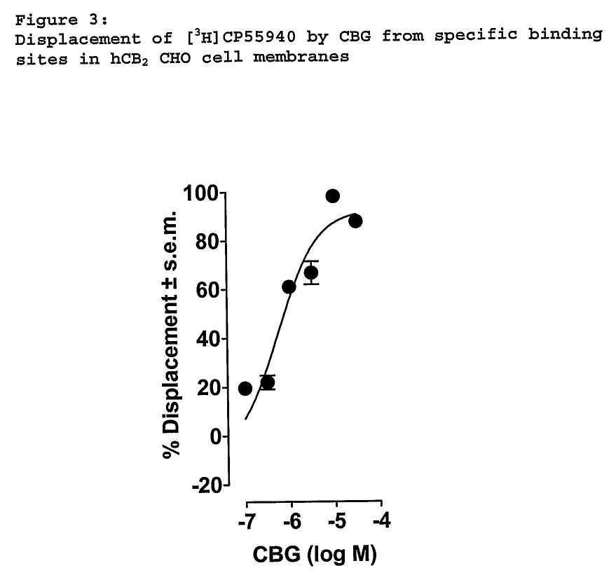

FIG. 3 shows a graph of displacement of [³H]CP55940 by CBG from specific binding sites in hCB₂ CHO cell membranes.

### EXAMPLE 1

### Investigation into the Properties of CBG at the CB₁ and CB₂ Receptors

The major constituent of cannabis delta-9-tetrahydrocannabinol (THC) has been well investigated as a medicinal substance yet its therapeutic usefulness can often be hindered by its additional psychotropic activity. This often limits the amount of THC that can be administered to a patient.

In contrast the naturally occurring plant cannabinoid cannabidiol (CBG) has been less well documented therapeutically, although it is known that CBG is non-psychoactive and has antimicrobial and anti-inflammatory properties.

The current study investigated the effects of CBG at the CB₁ and CB₂ receptors themselves. CB₁ receptors in mouse brain tissue and CB₂ receptors in CHO cell membranes transfected with human CB₂ receptors were used to compare the properties of CBD with the established CB₁ receptor and CB₂ receptor agonist CP55940.

### Method:

The test articles used were: CBG (purified plant extract), and CP55940. The compounds were dissolved in DMSO prior to use.

Whole mouse brain membranes were prepared as described by Thomas et al., 2004. CHO cells were stably transfected with cDNA encoding human cannabinoid CB₂ receptors and were maintained at 37° C. and 5% CO₂ in Dulbecco's Modified Eagle's Medium nutrient mixture.

### Radioligand Displacement Assay

The assays were carried out with the established CB₁ and CB₂ cannabinoid receptor agonist CP55940. This was radiolabelled to form [³H]CP55940.

Binding of the radiolabelled compound was initiated by the addition of either the brain membranes (33 μg protein per tube) or the transfected hCB₂ cells (25 μg protein per tube).

All assays were performed at 37° C. for 60 min before termination by addition of ice-cold wash buffer (50 mM Tris buffer, 1 mg ml⁻¹ bovine serum albumin, pH 7.4) and vacuum filtration using a 24-well sampling manifold and GF/B filters that had been soaked in wash buffer at 4° C. for at least 24 h.

[³⁵S]GTPγS Binding Assay

The assays were carried out with GTPγS binding buffer (50 mM Tris-HCl; 50 mM Tris-Base; 5 mM MgCl₂; 1 mM EDTA; 100 mM NaCl; 1 mM DTT; 0.1% BSA) in the presence of [³⁵S]GTPγS and GDP, in a final volume of 500 μl. Binding was initiated by the addition of [³⁵S]GTPγS to the tubes. The drugs were incubated in the assay for 60 min at 30° C. The reaction was terminated by a rapid vacuum filtration method using Tris buffer (50 mM Tris-HCl; 50 mM Tris-Base; 0.1% BSA), as described previously, and the radioactivity was quantified by liquid scintillation spectrometry.

The agonism of the CB₁ or CB₂ receptors by CP55940 results in a response in the cell. This response is the binding of [³⁵S]GTPγS to the cell membrane.

Changes in the response in the presence of the test compound can be measured in order to determine whether the compound is acting as an agonist, a neutral antagonist or an inverse agonist. An agonist will increase the response, a neutral antagonist will have no effect on the response and an inverse agonist will stop or reverse the response. The K_{B}-value that results from these investigations is therefore an indicator of the cells response.

The test compounds were also tested to determine whether they were able to displace the agonist CP55940 from the binding site of the CB₁ or CB₂ receptor. The Kᵢ-value that resulted from this investigation gives an insight into how strongly the test compound competes with the agonist for the binding site.

### Results:

It was shown that CBG can displace [³H]CP55940 from specific binding sites on mouse brain membranes (Kᵢ=439 nM) and stimulate binding of [³⁵S]GTPγS to these membranes, with an EC₅₀ of 0.05 nM as is shown by the graph in FIG. 2.

As shown in the graph in FIG. 3, CBG displaced [³H]CP55940 from specific binding sites on membranes prepared from hCB₂-CHO cells (Kᵢ=337 nM) and, at submicromolar concentrations, inhibited the ability of 5 μM forskolin to stimulate cyclic AMP production by these cells, albeit with an efficacy less than that of CP55940. The EC₅₀ was 388 nM with an Eₘₐₓ of 28.3.

To conclude the data presented in the example above show that CBG is a partial agonist at both the CB₁ and CB₂ cannabinoid receptors. As such this naturally occurring cannabinoid has real potential for use in the treatment or prevention of diseases benefiting from concurrent agonsim of the CB₁ and CB₂ cannabinoid receptor.

1. Use of the cannabinoid cannabigerol (CBG) in the manufacture of a medicament for use in the treatment of diseases and conditions benefiting from agonism of the CB₁ and/or the CB₂ cannabinoid receptors.

2. Use as claimed in claim 1, of the cannabinoid cannabigerol (CBG) in the manufacture of a medicament for use in the treatment of diseases and conditions benefiting from agonism of the CB₁ cannabinoid receptor.

3. Use as claimed in claim 1, of the cannabinoid cannabigerol (CBG) in the manufacture of a medicament for use in the treatment of diseases and conditions benefiting from agonism of the CB₂ cannabinoid receptor.

4. Use as claimed in claim 1, of the cannabinoid cannabigerol (CBG) in the manufacture of a medicament for use in the treatment of diseases and conditions benefiting from concurrent agonism of the CB₁ and the CB₂ cannabinoid receptors.

5. Use as claimed in any of claims 1 to 4, wherein the diseases or conditions to be treated are taken from the group: pain (including but not limited to acute pain; chronic pain; neuropathic pain and cancer pain), neurodegenerative disease (including but not limited to Alzheimer's disease; Parkinson's disease; amyotrophic lateral sclerosis; Huntington's disease; multiple sclerosis; frontotemporal dementia; prion disease; Lewy body dementia; progressive supranuclear palsy; vascular dementia; normal pressure hydrocephalus; traumatic spinal cord injury; HIV dementia; alcohol induced neurotoxicity; Down's syndrome; epilepsy or any other related neurological or psychiatric neurodegenerative disease), ischemic disease (including but not limited to stroke; cardiac ischemia; coronary artery disease; thromboembolism; myocardial infarction or any other ischemic related disease), brain injury or damage (including but not limited to traumatic brain injury is taken from the group: diffuse axonal injury; concussion; contusion; whiplash or any other traumatic head or brain injury), acquired brain injury (including but not limited to stroke; anoxic brain injury; hypoxic brain injury or any other acquired brain injury), age related inflammatory or autoimmune disease, cachexia (including related conditions such as AIDS wasting disease, weight loss associated with cancer, chronic obstructive pulmonary disease or infectious diseases such as tuberculosis), nausea and vomiting, glaucoma, movement disorders, rheumatoid arthritis, asthma, allergy, psoriasis, Crohn's disease, systemic lupus erythematosus, diabetes, cancer, osteoporosis, renal ischemia and nephritis.

6. Use as claimed in any of the preceding claims, wherein the cannabigerol is in the form of an extract prepared from at least one cannabis plant.

7. Use as claimed in any of the preceding claims, wherein the extract from at least one cannabis plant is a botanical drug substance.

8. Use as claimed in any of the preceding claims, wherein the extract from at least one cannabis plant is produced by extraction with supercritical or subcritical CO₂.

9. Use as claimed in any of the preceding claims, wherein the extract from at least one cannabis plant is produced by contacting plant material with a heated gas at a temperature which is greater than 100° C., sufficient to volatilise one or more of the cannabinoids in the plant material to form a vapour, and condensing the vapour to form an extract.

10. Use as claimed in any of the preceding claims, wherein the extract from at least one cannabis plant comprises all of the naturally occurring cannabinoids in the plant.

11. Use as claimed in claim 1, wherein the cannabigerol is in a substantially pure or isolated form

12. Use as claimed in claim 1, wherein the cannabigerol is in a synthetic form.

13. Use as claimed in any of the preceding claims, wherein the cannabigerol is formulated as a pharmaceutical composition further comprising one or more pharmaceutically acceptable carriers, excipients or diluents.

14. A method for the treatment or prevention of diseases benefiting from concurrent agonism of the CB₁ and the CB₂ cannabinoid receptors, which comprises administering to a subject in need thereof an effective amount of cannabigerol.
